(19) United States
(12) Patent Application Publication
(10) Pub. No.: US 2025/0347816 A1
(43) Pub. Date: Nov. 13, 2025

(54) Abnormality Detection in Movable Component in Optical Path

(71) Applicant: Siemens Healthineers AG, Forchheim (DE)

(72) Inventors: Wen Hao Chen, Shanghai (CN); Yi Tian, Shanghai (CN); Li Dong Zhang, Shanghai (CN); Liang Yan Fan, Shanghai (CN)

(73) Assignee: Siemens Healthineers AG, Forchheim (DE)

(21) Appl. No.: 19/201,015

(22) Filed: May 7, 2025

(30) Foreign Application Priority Data
May 10, 2024 (CN) 202410580198.9

(51) Int. Cl. G01T 7/00

(52) U.S. Cl. CPC G01T 7/00

(57) ABSTRACT

A method for detecting an abnormality in a movable component in an optical path. The method includes: acquiring a difference Diff(k, l, seg, r) between air correction charts with and without the movable component; determining a second difference DIFF′(k, l, r) according to the difference Diff(k, l, seg, r); subjecting the second difference DIFF′(k, l, r) to low-pass filtering to obtain a smooth signal S^{smooth}; suppressing the influence of module response difference according to the smooth signal S^{smooth} to obtain a reference signal S(k, l, r); subjecting S(k, l, r) to high-pass filtering in a channel direction to obtain a high-pass signal S^{HP}; comparing the high-pass signal S^{HP} with a threshold T, to determine an abnormality in the movable component.

In the present patent application, nouns and pronouns referring to people are not limited to a specific gender.

### TECHNICAL FIELD

The present disclosure relates to the detection of abnormalities in movable components in optical paths, in particular an optical path in a medical imaging device.

### BACKGROUND

In a computed tomography (CT) scan, the presence of abnormal components in the optical path will cause artifacts in the image. Examples of such abnormalities include processing defects and deformations caused by knocks. Existing methods of inspecting optical path components are either directed at immovable components or require additional scanning, perhaps even requiring the production of an image.

In addition, CT systems need to use an air correction chart to correct data obtained by scanning.

### SUMMARY

In view of the above, the present disclosure proposes a method for detecting an abnormality in a movable component in an optical path, a computer program, a computer-readable storage medium, and a medical imaging device.

According to a first aspect of the present disclosure, a method is provided for detecting an abnormality in a movable component in an optical path, the method comprising:
acquiring a difference Diff(k, l, seg, r) between air correction charts with and without the movable component, wherein k represents a channel, l represents a row, sec represents a partition, and r represents a focus position;
determining a second difference DIFF′(k, l, r) according to the difference Diff(k, l, seg, r);
subjecting the second difference DIFF′(k, l, r) to low-pass filtering to obtain a smooth signal S^{smooth};
suppressing the influence of module response difference according to the smooth signal S^{smooth} to obtain a reference signal S(k, l, r);
subjecting S(k, l, r) to high-pass filtering in a channel direction to obtain a high-pass signal S^{HP};
comparing the high-pass signal S^{HP} with a threshold T, to determine an abnormality in the movable component.

In an aspect, the abovementioned steps comprise determining the second difference DIFF′(k, l, r) according to the following formula: DIFF′(k, l, r)=Diff(k, l, seg, r).

In an aspect, the abovementioned steps comprise: averaging the difference Diff(k, l, seg, r) in a partition dimension to obtain the second difference DIFF′(k, l, r), DIFF′(k, l, r)=mean(Diff, 3), wherein mean(Diff, 3) represents averaging Diff in a third dimension.

In an aspect, the abovementioned steps comprise: subjecting the difference Diff(k, l, seg, r) to numerical translation to obtain the second difference DIFF′(k, l, r).

In an aspect, the movable component is an ultra-high resolution comb.

In an aspect, the abovementioned steps comprise subjecting the difference Diff(k, l, seg, r) to numerical translation according to the following formula:

$${DIFF}^{′} ( k , l , r ) = Diff ( k , l , seg , r ) + {\sum }_{i = k}^{N - 1} shift 1 ( k , r ) 1 \leq k \leq N - 1$$

$$wherein :$$

$$shift 1 ( k , r ) = \{ \begin{matrix}shift 0 ( k , r ) & abs ( shift 0 ( k , r ) ) > T 1 \\ 0 & else\end{matrix}$$

$$shift 0 ( k , r ) = \frac{1}{{N}_{i}} {\sum }_{l} ( Diff ( k + 1 , l , seg , r ) - Diff ( k , l , seg , r ) ) 1 \leq k \leq N - 1$$

N is the number of channels in the same row of a detector, Nₗis the number of rows, T1 is a threshold for determination, and abs( ) is an absolute value function.

In an aspect, the abovementioned steps comprise:
performing edge extension on the second difference DIFF′, performing low-pass filtering, and cutting off an extension point to obtain a low-pass second difference DIFF^{Med};
performing edge extension on the low-pass second difference DIFF^{Med}, performing low-pass filtering, and cutting off an extension point to obtain a smooth signal S^{smooth}.

In an aspect, the abovementioned steps comprise performing edge extension on the low-pass second difference DIFF^{Med} according to the following formula and performing low-pass filtering:

$${DIFF}_{Ext}^{Med} = Median ( Ext ( {DIFF}^{′} , E L 0 ) )$$

where Median( ) is a median function, and Ext( ) is an extension function, defined as follows:

$$Ext ( P , EL ) = \{ \begin{matrix}P ( EL - k + 1 , l , r ) & k = 1 : EL \\ P ( k - EL , l , r ) & k = EL + 1 : N + EL \\ P ( 2 * N + EL - k + 1 , l , r ) & k = N + EL + 1 : N + 2 * EL\end{matrix}$$

where N is the number of channels in the same row of the detector, and EL is an extension length.

In an aspect, the abovementioned steps comprise performing edge extension on the low-pass second difference DIFF^{Med} according to the following formula and performing low-pass filtering:

$${S}_{Ext 1} = Ext ( {DIFF}^{Med} , EL 1 )$$

$${S}_{Ext 1}^{s mooth} = LP ( {S}_{Ext 1} , Nw 1 , Ns 1 )$$

where LP (Q, Nw, Ns) is a low-pass function, defined as follows:

$$L P ( Q , Nw , Ns ) = iFFT ( ifftshift ( fftshift ( FFT ( Q ) ) . * W ) )$$

where FFT is a fast Fourier transform, iFFT is an inverse fast Fourier transform, fftshift is zero-frequency shift, ifftshift is inverse zero-frequency shift, and ·* is a dot product operation;
a filter function W is defined as follows:

$$W ( k ) = \{ \begin{matrix}0 & k < \frac{N}{2} + EL - Nw + 1 \\ conv ( k - ( \frac{N}{2} + EL + 1 ) ) / max ( conv ) & \begin{matrix}\frac{N}{2} + EL - Nw + 1 \leq \\ k \leq \frac{N}{2} + EL + Nw + 1\end{matrix} \\ 0 & k > \frac{N}{2} + EL + Nw + 1\end{matrix}$$

$$conv ( k ) = \frac{{e}^{- 0.5 * {( k * \frac{N s}{N w} )}^{2}}}{{\sum }_{i = - N w}^{i = N w} {e}^{- 0.5 * {( i * \frac{N s}{N w} )}^{2}}}$$

Ns and Nw are parameters of a Gaussian function conv(k);
EL1 is a concrete instance of EL, Ns1 is a concrete instance of Ns, and Nw1 is a concrete instance of Nw.

In an aspect, the abovementioned steps comprise obtaining the reference signal S(k, l, r) according to the following formula:

$$S ( k , l , r ) = \{ \begin{matrix}{S}^{smooth} ( k , l , r ) & 1 \leq k \leq CH \\ {S}^{smooth} ( k , l , r ) - shift 2 ( ceil ( \frac{k}{CH} - 1 ) , l , r ) & CH < k \leq N\end{matrix}$$

$$shift 2 ( m , l , r ) = {\sum }_{n = 1}^{m} ( {S}^{s mooth} ( CH \cdot n + 1 , l , r ) - {S}^{s mooth} ( CH \cdot n , l , r ) )$$

$$1 \leq m < \frac{N}{C H}$$

where CH is the number of channels in the same row of the detector in a module, and ceil ( ) is a ceiling function.

In an aspect, the abovementioned steps comprise obtaining the high-pass signal S^{HP} according to the following formula and steps:

$${S}_{Ext 2} = E x t ( S , EL 2 )$$

$${S}_{Ext 2}^{L P} = L P ( {S}_{Ext 2} , Nw 2 , Ns 2 )$$

cutting off an extension point from

$${S}_{Ext 2}^{L P}$$

to obtain a low-pass signal S^{lp};

$${S}^{HP} = S - {S}^{lp}$$

where Ext( ) is an extension function, defined as follows:

$$Ext ( P , EL ) = \{ \begin{matrix}P ( EL - k + 1 , l , r ) & k = 1 : EL \\ P ( k - EL , l , r ) & k = EL + 1 : N + EL \\ P ( 2 * N + EL - k + 1 , l , r ) & k = N + EL + 1 : N + 2 * EL\end{matrix}$$

where N is the number of channels in the same row of the detector, and EL is an extension length;
LP (Q, Nw, Ns) is a low-pass function, defined as follows:

$$LP ( Q , Nw , Ns ) = iFFT ( ifftshift ( fftshift ( FFT ( Q ) ) . * W ) )$$

where FFT is a fast Fourier transform, iFFT is an inverse fast Fourier transform, fftshift is zero-frequency shift, ifftshift is inverse zero-frequency shift, and ·* is a dot product operation;
a filter function W is defined as follows:

$$W ( k ) = \{ \begin{matrix}0 & k < \frac{N}{2} + EL - Nw + 1 \\ conv ( k - ( \frac{N}{2} + EL + 1 ) ) / max ( conv ) & \begin{matrix}\frac{N}{2} + EL - Nw + 1 \leq \\ k \leq \frac{N}{2} + EL + Nw + 1\end{matrix} \\ 0 & k > \frac{N}{2} + EL + Nw + 1\end{matrix}$$

$$conv ( k ) = \frac{{e}^{- 0.5 * {( k * \frac{N s}{N w} )}^{2}}}{{\sum }_{i = - N w}^{i = N w} {e}^{- 0.5 * {( i * \frac{N s}{N w} )}^{2}}}$$

where Ns and Nw are parameters of a Gaussian function conv(k);
EL2 is a concrete instance of EL, Ns2 is a concrete instance of Ns, and Nw2 is a concrete instance of Nw.

In an aspect, the threshold T is defined as follows:

$$T = \{ \begin{matrix}c & - f + d \leq k \leq f + d \\ {a}_{1} \sqrt{\vert k - d \vert} + {a}_{2} & else\end{matrix}$$

where

$${a}_{1} = \frac{b - c}{\sqrt{d - 1} - \sqrt{f}} , {a}_{2} = \frac{b \sqrt{f} - c \sqrt{d - 1}}{- \sqrt{d - 1} + \sqrt{f}} ,$$

b is a threshold of a detector edge, c is a threshold of a detector center, d is a channel ordinal number of the detector center, and f is the number of channels in the same row at the left and right of the detector center with the threshold c.

According to a second aspect of the present disclosure, a computer program is provided which, when executed by a processor, can realize the steps of the method described above.

According to a third aspect of the present disclosure, a computer-readable storage medium with a computer program stored thereon is provided, wherein the program, when executed by a processor, can realize the steps of the method described above.

According to a fourth aspect of the present disclosure, a medical imaging device with an X-ray tube is provided, comprising the computer-readable storage medium described above.

The method for detecting an abnormality in a movable component in an optical path, the computer program, the computer-readable storage medium, and the medical imaging device of the present disclosure utilize existing air correction charts to comprehensively investigate abnormalities in movable components, without the need for additional scanning or image reconstruction, so can effectively prevent image quality issues due to component abnormalities. The present disclosure can check movable components one by one, to precisely indicate which component has an abnormality. This will facilitate troubleshooting, reducing final testing times on production lines, and reducing production line costs.

### BRIEF DESCRIPTION OF THE DRAWINGS

Preferred aspects of the present disclosure will be described in detail below with reference to the drawings, to give an ordinary person skilled in the art a clearer understanding of the abovementioned and other features and advantages of the present disclosure. In the drawings:

FIG. 1 is a schematic flow chart of a method for detecting an abnormality in a movable component in an optical path according to an aspect of the present disclosure.

FIG. 2A is a schematic figure of an air correction chart with a movable component according to an aspect of the present disclosure.

FIG. 2B is a schematic figure of an air correction chart without a movable component according to an aspect of the present disclosure.

FIG. 2C is a schematic figure of the difference between the air correction chart of FIG. 2A and the air correction chart of FIG. 2B.

FIG. 3 is a schematic figure of a reference signal according to an aspect of the present disclosure.

FIG. 4 is a schematic figure of a threshold according to an aspect of the present disclosure.

FIG. 5 is a schematic figure of a high-pass signal and a threshold according to an aspect of the present disclosure.

### DETAILED DESCRIPTION

To clarify the objectives, technical solutions and advantages of the present disclosure, the present disclosure will be explained in further detail below through aspects.

An extension function Ext(P, EL) for extension of P in a channel direction is defined as follows:

$$Ext ( P , E L ) = \{ \begin{matrix}P ( E L − k + 1 , l , r ) & k = 1 : EL \\ P ( k − E L , l , r ) & k = E L + 1 : N + E L \\ P ( 2 ⋆ N + E L − k + 1 , l , r ) & k = N + E L + 1 : N + 2 ⋆ EL\end{matrix}$$

wherein, in P(k, l, r), k represents a channel, l represents a row, r represents a focus position, N is the number of channels in the same row of a detector, and EL is an extension length.

A low-pass function LP (Q, Nw, Ns) is defined as follows:

$$LP ( Q , Nw , Ns ) = iFFT ( ifftshift ( fftshift ( FFT ( Q ) ) . * W ) )$$

where FFT is a fast Fourier transform, iFFT is an inverse fast Fourier transform, fftshift is shifting a zero-frequency component to the center (zero-frequency shift), ifftshift is inverse zero-frequency shift, and ·* is a dot product operation. Matlab provides examples of fftshift and ifftshift, specifically:
Y=fftshift(X) rearranges a Fourier transform X by shifting the zero-frequency component to the center of an array.

If X is a vector, fftshift will exchange two halves (left and right) of X.

If X is a matrix, fftshift will exchange the first quadrant of X with the third quadrant, and exchange the second difference quadrant with the fourth quadrant.

If X is a multi-dimensional array, fftshift will exchange half-spaces of X along each dimension.

A filter function W is defined as follows:

$$W ( k ) = \{ \begin{matrix}0 & \begin{matrix}k < \frac{N}{2} + EL − \\ Nw + 1\end{matrix} \\ conv ( k − ( \frac{N}{2} + E L + 1 ) ) / max ( conv ) & \begin{matrix}\frac{N}{2} + E L − N w + 1 \leq k \leq \\ \frac{N}{2} + E L + Nw + 1\end{matrix} \\ 0 & k > \frac{N}{2} + E L + N w + 1\end{matrix}$$

$$conv ( k ) = \frac{{e}^{− 0.5 ⋆ {( k ⋆ \frac{N s}{N w} )}^{2}}}{{\sum }_{i = - Nw}^{i = Nw} {e}^{− 0.5 ⋆ {( i ⋆ \frac{N S}{N w} )}^{2}}}$$

Ns and Nw are parameters of a Gaussian function conv(k); Ns and Nw influence the waveform distribution of the Gaussian function conv(k).

In the present application, EL1 and EL2 are concrete instances of EL, Ns1 and Ns2 are concrete instances of Ns, and Nw1 and Nw2 are concrete instances of Nw.

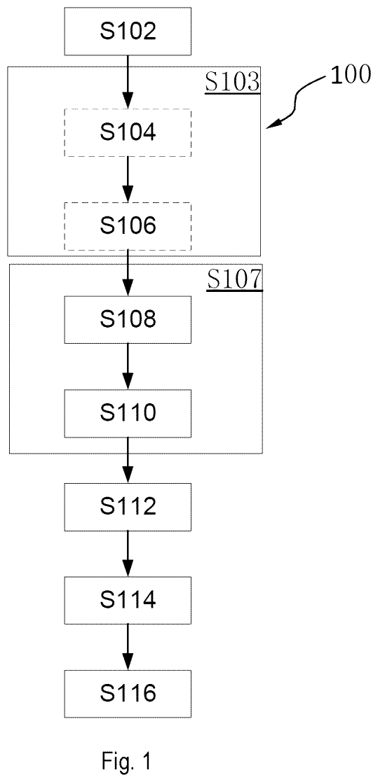

FIG. 1 is a schematic flow chart of a method 100 for detecting an abnormality in a movable component in an optical path according to an aspect of the present disclosure. The movable component is for example a movable wedge filter, split filter or UHR comb. As shown in FIG. 1, the method 100 for detecting an abnormality in a movable component in an optical path comprises step S102, step S103, step S107, step S112, step S114 and step S116.

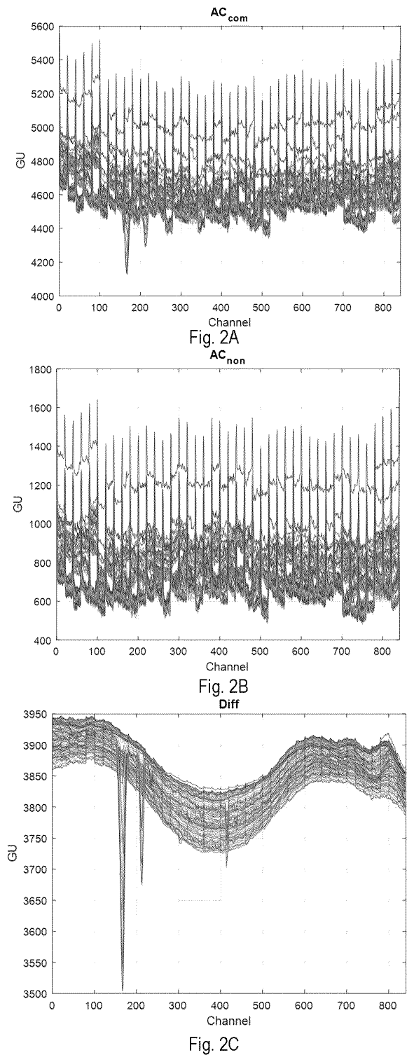

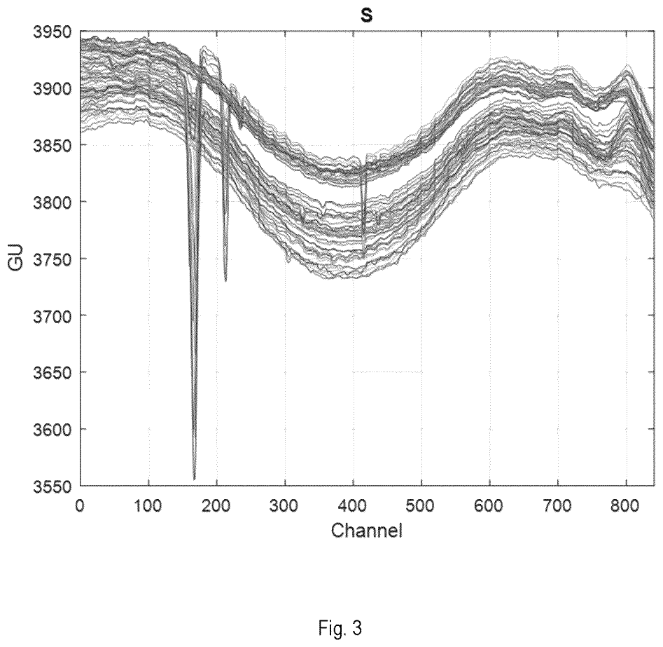

In step S102, the difference Diff(k, l, seg, r) between air correction charts with and without the movable component is acquired. FIG. 2A is a schematic figure of an air correction chart AC_{com}(k, l, seg, r) with a movable component according to an aspect of the present disclosure. FIG. 2B is a schematic figure of an air correction chart ACₙₒₙ(k, l, seg, r) without a movable component according to an aspect of the present disclosure. The air correction chart records a signal received by a detector in the course of an air scan. Here, k represents a channel, l represents a row, sec represents a partition, and r represents a focus position (focal spot index). For example, if there are two focus positions, then r=1 may be used to represent focus position 1, and r=2 may be used to represent focus position 2. A circumferential angle formed by rotation of the X-ray tube is evenly divided into partitions, and all angles within the same partition use an air correction chart of that partition. As shown in FIGS. 2A and 2B, different curves represent different rows. FIG. 2C is a schematic figure of the difference Diff(k, l, seg, r) between the air correction chart AC_{com}(k, l, seg, r) of FIG. 2A and the air correction chart ACₙₒₙ(k, l, seg, r) of FIG. 2B, wherein Diff(k, l, seg, r)=AC_{com}(k, l, seg, r)−ACₙₒₙ(k, l, seg, r). There is at least one air correction chart with a movable component and at least one air correction chart without a movable component in the X-ray optical path, and the difference Diff(k, l, seg, r) may even have already been calculated.

In step S103, a second difference DIFF′(k, l, r) is determined according to the difference Diff(k, l, seg, r). The second difference DIFF′(k, l, r) may be the same as the difference Diff(k, l, seg, r). In an aspect, optionally, before abnormality appraisal, to reduce noise, step S103 may further comprise step S104. In step S104, the difference Diff(k, l, seg, r) is averaged in a partition dimension to obtain the second difference DIFF′(k, l, r), DIFF′(k, l, r)=mean(Diff, 3), wherein mean(Diff, 3) represents averaging Diff in a third dimension (partition). This averaging operation is not a requirement; in the present application, for conciseness, DIFF′(k, l, r) may represent the average difference mean(Diff, 3) or the difference Diff(k, l, seg, r); these are collectively referred to as the second difference DIFF′(k, l, r).

To reduce normal signal jumping caused by filters such as an ultra-high resonance comb (UHR comb), step S103 may further comprise step S106. In step S106, the difference Diff(k, l, seg, r) is subjected to numerical translation (data shift) to obtain the second difference DIFF′(k, l, r). In this aspect, the expression for translation is;

$${DIFF}^{′} ( k , l , r ) = Diff ( k , l , seg , r ) + {\sum }_{i = k}^{N - 1} s h ift 1 ( k , r ) \qquad 1 \leq k \leq N - 1$$

$$wherein :$$

$$shift 1 ( k , r ) = \{ \begin{matrix}shift 0 ( k , r ) & abs ( shift 0 ( k , r ) ) > T 1 \\ 0 & else\end{matrix}$$

$$shift 0 ( k , r ) = \frac{1}{{N}_{l}} {\sum }_{l} ( Diff ( k + 1 , l , seg , r ) − Diff ( k , l , seg , r ) ) \qquad 1 \leq k \leq N − 1$$

N is the number of channels in the same row of a detector, Nₗ is the number of rows, T1 is a threshold for determination, and abs( ) is an absolute value function. Numerical translation can avoid misjudgements caused by the abovementioned normal signal jumping.

It should be noted that the formula above actually calculates DIFF′(k, l, sec, r), but for consistency of expression with other aspects, the partition variable is omitted.

In other aspects, depending on actual circumstances, numerical translation need not be performed. In other aspects, step S103 may simultaneously comprise step S104 and step S106, performed one after the other. In this case, it is only necessary to replace Diff(k, l, seg, r) on the right-hand side of the equation in step S106 with the result of step S104 (i.e. DIFF′(k, l, r)).

In step S107, the second difference DIFF′(k, l, r) is subjected to low-pass filtering to obtain a smooth signal S^{smooth}. In this aspect, step S107 may comprise step S108 and step S110.

Step S108: performing edge extension on the second difference DIFF′, performing low-pass filtering, and cutting off an extension point to obtain a low-pass second difference DIFF^{Med}. In this aspect, a 5-fold median filter may be used, represented as follows:

$${DIFF}_{Ext}^{M e d} = Median ( Ext ( {DIFF}^{′} , EL 0 ) )$$

It can be seen from the formula above that edge extension is performed on DIFF′ before median filtering, in order to keep the degrees of filtering of all points consistent.

An extension point is cut off from

$${DIFF}_{Ext}^{M e d}$$

to obtain a low-pass second difference DIFF^{Med}.

Step S110: performing edge extension on the low-pass second difference DIFF^{Med}, performing low-pass filtering, and cutting off an extension point to obtain a smooth signal S^{smooth}. In this aspect, the specific process is as follows:
Edge extension:

$${S}_{Ext 1} = E x t ( {DIFF}^{M e d} , EL 1 )$$

Low-pass filtering. In this aspect, low-pass filtering is performed according to the following formula:

$${S}_{Ext 1}^{smooth} = L P ( {S}_{Ext 1} , Nw 1 , Ns 1 )$$

In other aspects, a different low-pass filter may be used.

An extension point is cut off to obtain a smooth signal S^{smooth}.

Step S112: suppressing the influence of module response difference to obtain a reference signal S. In step S112, the following operation may be performed:

$$S ( k , l , r ) = \{ \begin{matrix}{S}^{smooth} ( k , l , r ) & 1 \leq k \leq C H \\ {S}^{smooth} ( k , l , r ) − shift 2 ( ceil ( \frac{k}{C H} − 1 ) , l , r ) & C H < k \leq N\end{matrix}$$

$$\begin{matrix}shift 2 ( m , l , r ) & = {\sum }_{n = 1}^{m} ( {S}^{smooth} ( CH \cdot n + 1 , l , r ) − {S}^{smooth} ( CH \cdot n , l , r ) )\end{matrix} \qquad 1 \leq m < \frac{N}{C H}$$

where CH is the number of channels in the same row in a module of the detector, and ceil ( ) is a ceiling function. S is a standard for detecting a component abnormality, so can be called a reference signal.

In S(k, l, r), only the high-pass part indicates a component abnormality. Therefore, in step S114, S(k, l, r) is subjected to high-pass filtering in a channel direction to obtain a high-pass signal S^{HP}. In this aspect, implementation is as follows:

$${S}_{Ext 2} = E x t ( S , EL 2 )$$

$${S}_{Ext 2}^{L P} = L P ( {S}_{Ext 2} , Nw 2 , Ns 2 )$$

An extension point is cut off from

$${S}_{Ext 2}^{L P}$$

to obtain a low-pass signal S^{lP}. Thus, the high-pass signal S^{HP} is calculated as follows:

$${S}^{HP} = S - {S}^{lp}$$

In step S116, the high-pass signal S^{HP} is compared with a threshold T, to determine abnormalities in movable components. These abnormalities may include the presence or absence of an abnormality, whether an abnormality is acceptable, etc. The correlation of signal error to image quality varies with the square root of the distance to the center of the detector. Thus, in this aspect, to reflect this phenomenon, a threshold T is defined as follows:

$$T = \{ \begin{matrix}c & − f + d \leq k \leq f + d \\ {a}_{1} \sqrt{\vert k − d \vert} + {a}_{2} & else\end{matrix}$$

where

$${a}_{1} = \frac{b − c}{\sqrt{d − 1} − \sqrt{f}} , {a}_{2} = \frac{b \sqrt{f} − c \sqrt{d − 1}}{− \sqrt{d − 1} + \sqrt{f}} ,$$

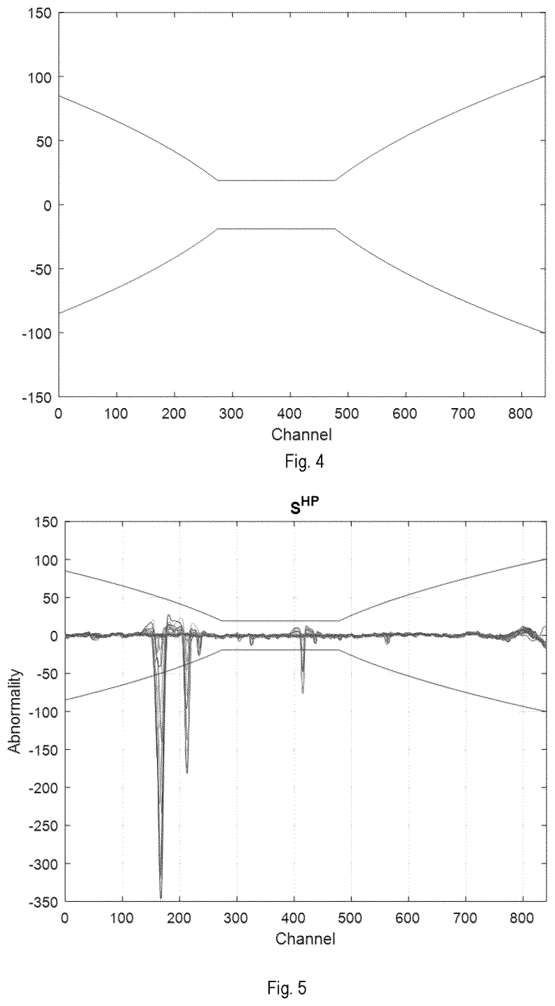

b is threshold of a detector edge, c is a threshold of a detector center, d is a channel ordinal number of the detector center, and f is the number of channels in the same row at the left and right of the detector center with the threshold c. A channel with threshold c has total length 2f. If an abnormality represented by the high-pass signal S^{HP} exceeds the threshold T, a warning is given that there is a high risk of image artifacts occurring due to a component abnormality. In other aspects, the threshold T may have other definitions. FIG. 4 is a schematic figure of a threshold T according to an aspect of the present disclosure. FIG. 5 is a schematic figure of a high-pass signal S^{HP} and a threshold T according to an aspect of the present disclosure.

Before comparison with the threshold T: in the case of UHR mode, a constant weighting factor w1 will multiply the high-pass signal: in the case of a split filter, another weighting operation may be performed:

$${S}_{w}^{HP} = {S}^{HP} ( k , l , r ) \cdot w ( l )$$

The objective of the abovementioned weighting operation is to enable different movable components to use the same threshold T. Of course, if different thresholds are set for different movable components, then weighting need not be performed.

According to a second aspect of the present disclosure, a computer program is provided which, when executed by a processor, can realize the steps of the method 100 for detecting an abnormality in a movable component in an optical path.

According to a third aspect of the present disclosure, a computer-readable storage medium with a computer program stored thereon is provided, wherein the program, when executed by a processor, can realize the steps of the method 100 for detecting an abnormality in a movable component in an optical path.

According to a fourth aspect of the present disclosure, a medical imaging device with an X-ray tube is provided, comprising the computer-readable storage medium described above.

The method for detecting an abnormality in a movable component in an optical path, the computer program, the computer-readable storage medium, and the medical imaging device of the present disclosure utilize existing air correction charts to comprehensively investigate abnormalities in movable components, without the need for additional scanning or image reconstruction, so can effectively prevent image quality issues due to component abnormalities. The present disclosure can check movable components one by one, to precisely indicate which component has an abnormality. This will facilitate troubleshooting, reducing final testing times on production lines, and reducing production line costs.

The above are merely preferred aspects of the present disclosure, which are not intended to limit it. Any amendments, equivalent substitutions or improvements etc. made within the spirit and principles of the present disclosure shall be included in the scope of protection thereof.

1. A method for detecting an abnormality in a movable component in an optical path, the method comprising:
step S102, acquiring a difference Diff(k, l, seg, r) between air correction charts with and without the movable component, wherein k represents a channel, l represents a row, sec represents a partition, and r represents a focus position;
step S103, determining a second difference DIFF′(k, l, r) according to the difference Diff(k, l, seg, r);
step S107, subjecting the second difference DIFF′(k, l, r) to low-pass filtering to obtain a smooth signal S^{smooth};
step S112, suppressing the influence of module response difference according to the smooth signal S^{smooth} to obtain a reference signal S(k, l, r);
step S114, subjecting the reference signal S(k, l, r) to high-pass filtering in a channel direction to obtain a high-pass signal S^{HP}; and
step S116, comparing the high-pass signal S^{HP} with a threshold T, to determine an abnormality in the movable component.

2. The method according to claim 1, wherein step S103 comprises determining the second difference DIFF′(k, l, r) according to the following formula: DIFF′(k, l, r)=Diff(k, l, seg, r).

3. The method according to claim 1, wherein step S103 comprises:
step S104, averaging the difference Diff(k, l, seg, r) in a partition dimension to obtain the second difference DIFF′(k, l, r), DIFF′(k, l, r)=mean(Diff, 3), wherein mean(Diff, 3) represents averaging Diff in a third dimension.

4. The method according to claim 1, wherein step S103 comprises:
step S106: subjecting the difference Diff(k, l, seg, r) to numerical translation to obtain the second difference DIFF′(k, l, r).

5. The method according to claim 4, wherein the movable component is an ultra-high resolution comb.

6. The method according to claim 4, wherein step S106 comprises subjecting the difference Diff(k, l, seg, r) to numerical translation according to the following formula:
$${DIFF}^{′} ( k , l , r ) = Diff ( k , l , seg , r ) + {\sum }_{i = k}^{N - 1} shift 1 ( k , r ) \qquad 1 \leq k \leq N - 1$$
$$wherein :$$
$$shift 1 ( k , r ) = \{ \begin{matrix}shift 0 ( k , r ) & abs ( shift 0 ( k , r ) ) > T 1 \\ 0 & else\end{matrix}$$
$$shift 0 ( k , r ) = \frac{1}{{N}_{l}} {\sum }_{l} ( Diff ( k + 1 , l , seg , r ) − Diff ( k , l , seg , r ) ) \text{} \qquad 1 \leq k \leq N − 1$$
N is the number of channels in the same row of a detector, Nₗis the number of rows, T1 is a threshold for determination, and abs( ) is an absolute value function.

7. The method according to claim 1, wherein step S107 comprises step S108 and step S110;
step S108: performing edge extension on the second difference DIFF′, performing low-pass filtering, and cutting off an extension point to obtain a low-pass second difference DIFF^{Med}; and
step S110: performing edge extension on the low-pass second difference DIFF^{Med}, performing low-pass filtering, and cutting off an extension point to obtain a smooth signal S^{smooth}.

8. The method according to claim 7, wherein step S108 comprises performing edge extension on the low-pass second difference DIFF^{Med} according to the following formula and performing low-pass filtering:
$${DIFF}_{Ext}^{M e d} = Median ( E x t ( {DIFF}^{′} , EL 0 ) )$$
where Median( ) is a median function, and Ext( ) is an extension function, defined as follows:
$$Ext ( P , E L ) = \{ \begin{matrix}P ( E L − k + 1 , l , r ) & k = 1 : EL \\ P ( k − E L , l , r ) & k = E L + 1 : N + E L \\ P ( 2 ⋆ N + E L − k + 1 , l , r ) & k = N + E L + 1 : N + 2 ⋆ EL\end{matrix}$$
where N is the number of channels in the same row of the detector, and EL is an extension length.

9. The method according to claim 8, wherein step S110 comprises performing edge extension on the low-pass second difference DIFF^{Med} according to the following formula and performing low-pass filtering:
$${S}_{Ext 1} = E x t ( {DIFF}^{M e d} , EL 1 )$$
$${S}_{Ext 1}^{smooth} = L P ( {S}_{Ext 1} , Nw 1 , Ns 1 )$$
$$where LP ( Q , Nw , Ns ) is a low - pass function , defined as follows : LP ( Q , Nw , Ns ) = iFFT ( ifftshift ( fftshift ( FFT ( Q ) ) . * W ) )$$
where FFT is a fast Fourier transform, iFFT is an inverse fast Fourier transform, fftshift is zero-frequency shift, ifftshift is inverse zero-frequency shift, and ·* is a dot product operation;
a filter function W is defined as follows:
$$W ( k ) = \{ \begin{matrix}0 & \begin{matrix}k < \frac{N}{2} + EL − \\ Nw + 1\end{matrix} \\ conv ( k − ( \frac{N}{2} + E L + 1 ) ) / max ( conv ) & \begin{matrix}\frac{N}{2} + E L − N w + 1 \leq k \leq \\ \frac{N}{2} + E L + N w + 1\end{matrix} \\ 0 & k > \frac{N}{2} + E L + N w + 1\end{matrix}$$
$$conv ( k ) = \frac{{e}^{− 0.5 ⋆ {( k ⋆ \frac{N s}{N w} )}^{2}}}{{\sum }_{i = − Nw}^{i = Nw} {e}^{− 0.5 ⋆ {( i ⋆ \frac{N S}{N w} )}^{2}}}$$
a Gaussian function conv(k); and
EL1 is a concrete instance of EL, Ns1 is a concrete instance of Ns, and Nw1 is a concrete instance of Nw.

10. The method according to claim 1, wherein step S112 comprises obtaining the reference signal S(k, l, r) according to the following formula:
$$S ( k , l , r ) \qquad = \{ \begin{matrix}{S}^{smooth} ( k , l , r ) & 1 \leq k \leq C H \\ {S}^{smooth} ( k , l , r ) − shift 2 ( ceil ( \frac{k}{C H} − 1 ) , l , r ) & C H < k \leq N\end{matrix}$$
$$\begin{matrix}shift 2 ( m , l , r ) & = {\sum }_{n = 1}^{m} ( {S}^{smooth} ( CH \cdot n + 1 , l , r ) − {S}^{smooth} ( CH \cdot n , l , r ) )\end{matrix} \qquad \text{} 1 \leq m < \frac{N}{C H}$$
where CH is the number of channels in the same row in a module of the detector, and ceil ( ) is a ceiling function.

11. The method according to claim 1, wherein step S114 comprises obtaining the high-pass signal S^{HP} according to the following formula and steps:
$${S}_{Ext 2} = E x t ( S , EL 2 )$$
$${S}_{Ext 2}^{L P} = L P ( {S}_{Ext 2} , Nw 2 , Ns 2 )$$
cutting off an extension point from
$${S}_{Ext 2}^{L P}$$
to obtain a low-pass signal S^{lP};
$${S}^{H P} = S - {S}^{lp}$$
where Ext( ) is an extension function, defined as follows:
$$Ext ( P , E L ) = \{ \begin{matrix}P ( E L − k + 1 , l , r ) & k = 1 : EL \\ P ( k − E L , l , r ) & k = E L + 1 : N + E L \\ P ( 2 ⋆ N + E L − k + 1 , l , r ) & k = N + E L + 1 : N + 2 ⋆ EL\end{matrix}$$
where N is the number of channels in the same row of the detector, and EL is an extension length;
LP (Q, Nw, Ns) is a low-pass function, defined as follows:
$$LP ( Q , Nw , Ns ) = i F F T ( ifftshift ( fftshift ( F F T ( Q ) ) . * W ) )$$
where FFT is a fast Fourier transform, iFFT is an inverse fast Fourier transform, fftshift is zero-frequency shift, ifftshift is inverse zero-frequency shift, and ·* is a dot product operation;
a filter function W is defined as follows:
$$W ( k ) = \{ \begin{matrix}0 & \begin{matrix}k < \frac{N}{2} + EL − \\ Nw + 1\end{matrix} \\ conv ( k − ( \frac{N}{2} + E L + 1 ) ) / max ( conv ) & \begin{matrix}\frac{N}{2} + E L − N w + 1 \leq k \leq \\ \frac{N}{2} + E L + N w + 1\end{matrix} \\ 0 & k > \frac{N}{2} + E L + N w + 1\end{matrix}$$
$$conv ( k ) = \frac{{e}^{− 0.5 ⋆ {( k ⋆ \frac{N s}{N w} )}^{2}}}{{\sum }_{i = − Nw}^{i = Nw} {e}^{− 0.5 ⋆ {( i ⋆ \frac{N S}{N w} )}^{2}}}$$
where Ns and Nw are parameters of a Gaussian function conv(k);
EL2 is a concrete instance of EL, Ns2 is a concrete instance of Ns, and Nw2 is a concrete instance of Nw.

12. The method according to claim 1, wherein the threshold T is defined as follows:
$$T = \{ \begin{matrix}c & - f + d \leq k \leq f + d \\ {a}_{1} \sqrt{\vert k - d \vert} + {a}_{2} & else\end{matrix}$$
where
$${a}_{1} = \frac{b − c}{\sqrt{d − 1} − \sqrt{f}} , {a}_{2} = \frac{b \sqrt{f} − c \sqrt{d − 1}}{− \sqrt{d − 1} + \sqrt{f}} ,$$
b is a threshold of a detector edge, c is a threshold of a detector center, d is a channel ordinal number of the detector center, and f is the number of channels in the same row at the left and right of the detector center with the threshold c.

13. A non-transitory computer-readable storage medium with a computer program stored thereon, wherein the program, when executed by a processor, performs the following steps:
step S102, acquiring a difference Diff(k, l, seg, r) between air correction charts with and without the movable component, wherein k represents a channel, l represents a row, sec represents a partition, and r represents a focus position;
step S103, determining a second difference DIFF′(k, l, r) according to the difference Diff(k, l, seg, r);
step S107, subjecting the second difference DIFF′(k, l, r) to low-pass filtering to obtain a smooth signal S^{smooth};
step S112, suppressing the influence of module response difference according to the smooth signal S^{smooth} to obtain a reference signal S(k, l, r);
step S114, subjecting the reference signal S(k, l, r) to high-pass filtering in a channel direction to obtain a high-pass signal S^{HP}; and
step S116, comparing the high-pass signal S^{HP} with a threshold T, to determine an abnormality in the movable component.

14. A medical imaging device with an X-ray tube, the medical imaging device comprising the non-transitory computer-readable storage medium according to claim 13.

15. The medical imaging device according to claim 14, wherein step S103 comprises determining the second difference DIFF′(k, l, r) according to the following formula:
DIFF′(k, l, r)=Diff(k, l, seg, r).

16. The medical imaging device according to claim 15, wherein step S103 comprises:
step S104, averaging the difference Diff(k, l, seg, r) in a partition dimension to obtain the second difference DIFF′(k, l, r), DIFF′(k, l, r)=mean(Diff, 3), wherein mean(Diff, 3) represents averaging Diff in a third dimension.

17. The medical imaging device according to claim 15, wherein step S103 comprises:
step S106: subjecting the difference Diff(k, l, seg, r) to numerical translation to obtain the second difference DIFF′(k, l, r).

18. The medical imaging device according to claim 15, wherein step S107 comprises:
step S108: performing edge extension on the second difference DIFF′, performing low-pass filtering, and cutting off an extension point to obtain a low-pass second difference DIFF^{Med}; and
step S110: performing edge extension on the low-pass second difference DIFF^{Med}, performing low-pass filtering, and cutting off an extension point to obtain a smooth signal S^{smooth}.

19. The medical imaging device according to claim 15, wherein step S112 comprises obtaining the reference signal S(k, l, r) according to the following formula:
$$S ( k , l , r ) \qquad = \{ \begin{matrix}{S}^{smooth} ( k , l , r ) & 1 \leq k \leq C H \\ {S}^{smooth} ( k , l , r ) − shift 2 ( ceil ( \frac{k}{C H} − 1 ) , l , r ) & C H < k \leq N\end{matrix}$$
$$\begin{matrix}shift 2 ( m , l , r ) & = {\sum }_{n = 1}^{m} ( {S}^{smooth} ( CH \cdot n + 1 , l , r ) − {S}^{smooth} ( CH \cdot n , l , r ) )\end{matrix} \qquad \text{} 1 \leq m < \frac{N}{C H}$$
where CH is the number of channels in the same row in a module of the detector, and ceil ( ) is a ceiling function.
